(19) United States
(12) Patent Application Publication
(10) Pub. No.: US 2026/0335369 A1
(43) Pub. Date: Oct. 29, 2026

(54) METHOD FOR PREPARING EXOSOME POLYPEPTIDE HYDROGEL AND USE THEREOF

(71) Applicant: Institute of Radiation Medicine, Chinese Academy of Medical Sciences, Tianjin (CN)

(72) Inventors: Ningning He, Tianjin (CN); Qiang Liu, Tianjin (CN); Huijuan Song, Tianjin (CN); Chang Xu, Tianjin (CN); Ning Wang, Tianjin (CN); Tong An, Tianjin (CN); Daqin Li, Tianjin (CN); Runxuan Wang, Tianjin (CN)

(21) Appl. No.: 19/223,010

(22) Filed: May 29, 2025

(30) Foreign Application Priority Data
Apr. 25, 2025 (CN) 202510525496.2

(51) Int. Cl. A61K 35/28; A61K 9/08; A61K 47/34; A61P 37/06

(52) U.S. Cl. CPC A61K 35/28; A61K 9/08; A61K 47/34; A61P 37/06

(57) ABSTRACT

Disclosed is a method for preparing an exosome polypeptide hydrogel and use thereof, which belongs to the technical field of pharmaceutical preparations. The specific method includes the following steps: obtaining an exosome solution; preparation of an oxidized methoxypolyethylene glycol-poly L-tyrosine copolymer Pet; mixing the exosome solution with the Pet to obtain an exosome polypeptide hydrogel. The exosome polypeptide hydrogel prepared by the present disclosure can activate the MFGE8-ERK signaling pathway to induce M2 polarization of macrophages, and is used to treat radiation-induced intestinal injury.

### CROSS-REFERENCE TO RELATED APPLICATION

This application claims priority of Chinese Patent Application No. 202510525496.2, filed on Apr. 25, 2025, the entire contents of which are incorporated herein by reference.

### TECHNICAL FIELD

The present disclosure belongs to the technical field of pharmaceutical preparations, and particularly relates to a method for preparing an exosome polypeptide hydrogel and use thereof.

### BACKGROUND

With the rapid development of modern radiotherapy technology, the number of patients with radiation-induced intestinal injury caused by abdominal tumor radiotherapy or accidental radiation exposure is steadily increasing. The radiotherapy technology leads to some related complications while improving the survival rate of patients, causing huge economic burden on patients and having a serious impact on their quality of life. The most common complication is radiation enteritis, which is caused by radiotherapy causing damage to normal intestinal cells, triggering the release of inflammatory factors, further leading to intestinal mucosal atrophy and ulceration, hindering the renewal of underlying epithelial cells, and even cell death. Therefore, the complex situation of radiation-induced intestinal injury poses an important challenge to existing treatment methods. Currently, clinically available radioprotective drugs have obvious limitations in the intestine; there are concerns about the off-target effects of pH-sensitive, ROS-sensitive, or receptor-mediated targeted nanoparticle-delivered drugs. To address these issues, a large number of studies have been conducted on precise drug delivery systems for radiation-induced intestinal injury, and mesenchymal stem cell-derived exosomes (MSC-Exo) have shown good effects in the treatment of a variety of diseases. Due to the influence of the gastrointestinal environment, the cost of Exo treatment is greatly increased, and their efficiency of action and effective targeted delivery remain a challenge. Therefore, it is generally agreed that designing new carriers for the targeted delivery of Exo therapeutic drugs for radiation-induced intestinal injury has great potential.

### SUMMARY

Polytyrosine polypeptide hydrogel (Pet) has acid resistance and a three-dimensional network structure. The present disclosure designs and constructs an orally administrable exosome polypeptide hydrogel (Pet-Exo), and loads exosomes (Exo) extracted from the supernatant of human umbilical cord-derived mesenchymal stem cells (hMSCs) into the polytyrosine polypeptide hydrogel to improve the bioavailability of exosomes and treat radiation-induced intestinal injury.

To achieve the above object, the present disclosure adopts the following technical solutions.

In a first aspect, the present disclosure provides a method for preparing an exosome polypeptide hydrogel, including the following steps:

S1, obtaining an exosome solution;

S2, preparation of an oxidized methoxypolyethylene glycol-poly L-tyrosine copolymer Pet: dissolving mPEG-NH₂in anhydrous DMF under protection of inert gas, then adding L-Tyr-NCAs, reacting at 30-50° C for 48-96 hours, after precipitation, filtration, and drying, adding water to dissolve, then dialyzing in water for 24-72 hours using a dialysis bag, freeze-drying after dialysis to obtain a copolymer PETyr, reacting the copolymer with tyrosinase for 24-96 hours to obtain Pet;

S3, mixing the exosome solution obtained in the step S1 with the Pet in the step S2 in a ratio of 1-2:1-2 to obtain an exosome polypeptide hydrogel.

Further, an exosome in the exosome solution in the step S1 is derived from mesenchymal stem cells.

Further, the exosome concentration in the exosome solution is 3 mg/mL.

Further, the inert gas in the step S2 is argon.

Further, the step S2 includes: dissolving 400 mg of mPEG-NH₂in 10 mL of anhydrous DMF, then adding 289 mg of L-Tyr-NCAs, reacting at 30° vC for 72 hours, after precipitation, filtration, and drying, adding water to dissolve, then dialyzing for 24-72 hours using a dialysis bag with a molecular weight cutoff of 1,000-2,000 Da, freeze-drying to obtain a copolymer PETyr, reacting the copolymer with tyrosinase for 48 hours to obtain Pet.

Further, a molecular weight of the mPEG-NH₂ is 2000 g/moL.

Further, a molecular weight cutoff of the dialysis bag is 1,000 Da and a dialysis time is 48 hours.

Further, a dosage ratio of the exosome solution to the Pet in the step S3 is 1:1.

In a second aspect, the present disclosure provides an exosome polypeptide hydrogel prepared by the method for preparing an exosome polypeptide hydrogel.

In a third aspect, the present disclosure further provides use of the exosome polypeptide hydrogel in the preparation of a product for treating radiation-induced intestinal injury.

Compared with the prior art, the present disclosure has the following beneficial effects.

The exosome polypeptide hydrogel prepared by the present disclosure can activate the MFGE8-ERK signaling pathway to induce M2 polarization of macrophages, and is used to treat radiation-induced intestinal injury. HE staining results show that the intestinal epithelial structure and intestinal villi are intact in the Pet-Exo group, and the crypt structure is clear, which alleviates the phenomenon of intestinal villi loss and incomplete epithelial structure caused by IR; Pet-Exo can promote the proliferation of intestinal cells after radiation damage and reduce the oxidative damage that may be caused by the increase of radiation-induced ROS; Pet-Exo exerts anti-inflammatory effects by inhibiting the polarization of macrophages to M1 macrophages and promoting the reprogramming of macrophages to M2 macrophages.

### BRIEF DESCRIPTION OF THE DRAWINGS

FIG. 1A is a SEM image of exosomes, scale: 200 nm;

FIG. 1B shows the Western blot detection of marker proteins in exosomes in Example 1;

FIG. 1C shows the particle size distribution of exosomes obtained by nanoparticle tracking analysis (NTA) in Example 1;

FIG. 1D shows the rheological measurements of the storage modulus G’ and loss modulus G’’ of Pet and Pet-Exo at different times in Example 1;

FIG. 1E shows the preparation of Pet by NCA ring-opening reaction in Example 1;

FIG. 1F shows the rheological properties of Pet in the angular frequency range of 0.1-100 rad/s in Example 1;

FIG. 1G shows the secondary structure of Pet in Example 1;

FIG. 1H shows the rheological measurements of the storage modulus G’ and loss modulus G’’ of Pet and Pet-Exo at different strains in Example 1;

FIG. 1I is a scanning electron microscope (SEM) image of Pet hydrogel in Example 1;

FIG. 1J shows the Pet-Exo hydrogel state and the SEM images of Pet and Pet-Exo in Example 1;

FIG. 2A is a fluorescence image of exosomes in mice at different time points after oral administration in Example 2;

FIG. 2B is a fluorescent image of the gastrointestinal tract of mice after oral administration in Example 2;

FIG. 3A shows confocal fluorescence images of bone marrow-derived macrophages labeled with M2 biomarkers CD206 (red) and F4/80 (green), with CD206 in red and F4/80 in green;

FIG. 3B shows confocal fluorescence images of macrophages labeled with M1 biomarkers iNOS (red) and F4/80 (green) in Example 3, with iNOS in red and F4/80 in green;

FIG. 4 shows apparent images of intestinal injury in mice and HE-stained cross-sectional structural diagrams of the small intestine in Example 4 (the red circled part shows inflammatory symptoms of edema);

FIG. 5 shows immunofluorescence staining images of Ki67 and ROS in small intestine tissues in Example 5;

FIG. 6A-B are immunofluorescence staining images of macrophages in the intestinal tissues of mice in Example 6;

FIG. 6C shows the expression of pro-inflammatory and anti-inflammatory factors in the serum of mice in different treatment groups in Example 6;

FIG. 7A shows the co-localization image of CD206 and exosome fluorescence in M2 macrophages in different treatment groups in Example 7; and

FIG. 7B shows the Western Blot detection of p-ERK and p-STAT3 protein expression levels in macrophages in each group in Example 7.

### DETAILED DESCRIPTION

In order to better illustrate the present disclosure, the following embodiments are given. Obviously, embodiments described are only a part of embodiments of the present disclosure, and are not all of embodiments thereof. All other embodiments obtained by those skilled in the art based on the embodiments of the present disclosure without creative efforts shall fall within the scope of protection of the present disclosure.

The technical solutions of the present disclosure are further described below in conjunction with accompanying drawings and embodiments.

Example 1 Preparation and characterization of exosome polytyrosine polypeptide hydrogel

### 1. Extraction and characterization of exosomes

Mesenchymal stem cells (MSCs) were isolated from human umbilical cord and cultured in a DMEM/F-12 medium system. The collected culture supernatant of mesenchymal stem cells was centrifuged at 300×g for 10 minutes at 4° C, the supernatant was aspirated, then centrifuged at 2 000×g for 10 minutes, then the supernatant was aspirated, and then centrifuged at 10,000×g for 30 minutes, and the supernatant was aspirated; ultracentrifugation was performed at 140,000 × g for 90 minutes. All centrifugations were performed at 4° C; the supernatant was removed, and the obtained precipitate was the exosomes. The precipitate was washed with PBS buffer and resuspended. The final exosome concentration was 3 mg/mL.

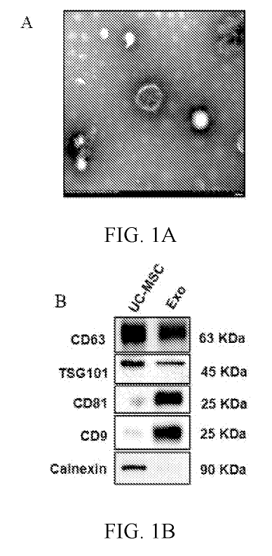

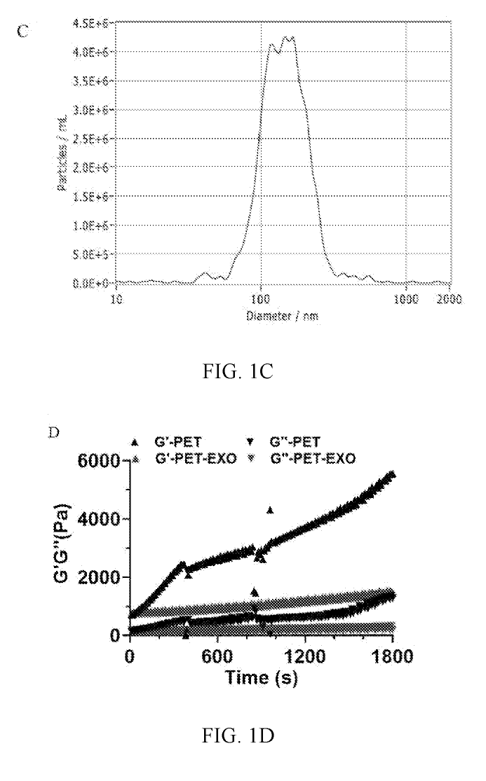

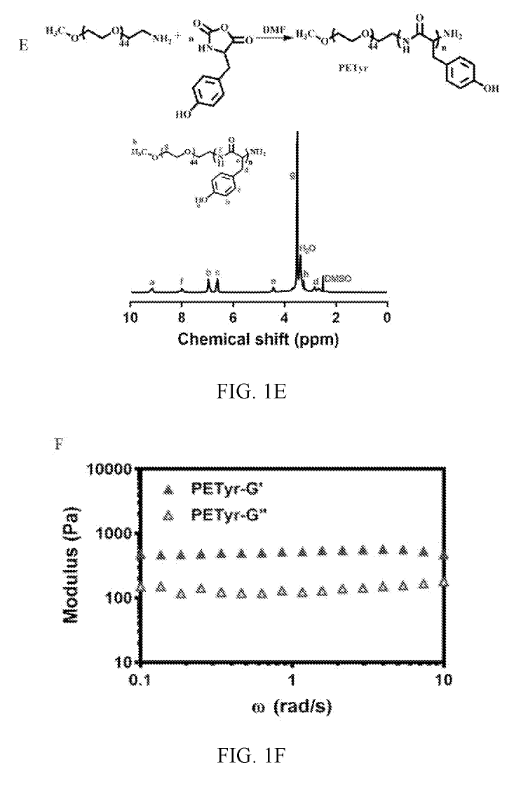

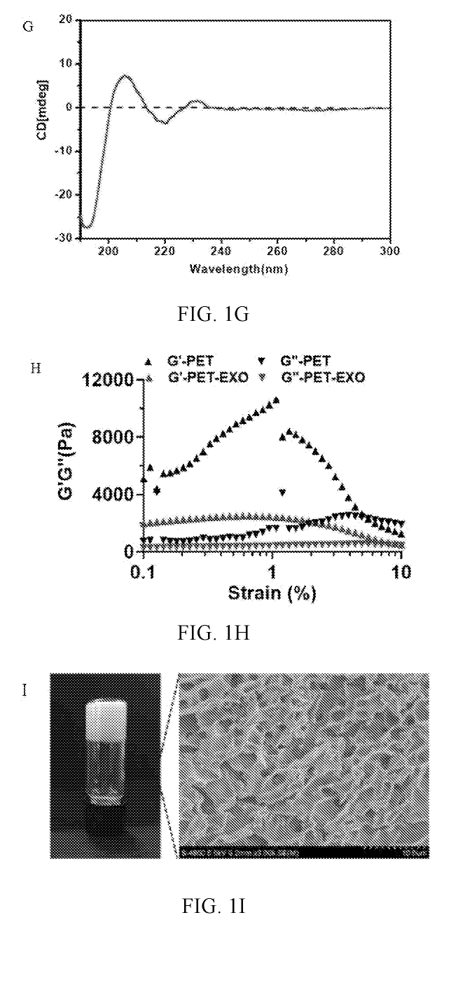

The obtained exosomes were characterized by transmission electron microscopy (TEM), nanoparticle tracking analyzer (NTA) and Western Blot. Under transmission electron microscopy, Exo had a discernible double-layer membrane structure, showing a typical cup-shaped or spherical morphology (FIG. 1A); Western Blot showed protein markers associated with Exo expression (FIG. 1B); NTA showed that the particle size of Exo was mainly distributed around 180 nm (FIG. 1C);

### 2. Preparation of oxidized methoxypolyethylene glycol-poly L-tyrosine copolymer (Pet)

First, methoxypolyethylene glycol amine (mPEG-NH2) was used as a macromolecular initiator, and L-tyrosine-N-carboxyl cyclic anhydride (L-Tyr-NCAs) ring-opening polymerization (ROP) was adopted to prepare methoxypolyethylene glycol-poly L-tyrosine amphiphilic copolymer PETyr, which was then reacted with tyrosinase to obtain the oxidized methoxypolyethylene glycol-poly L-tyrosine copolymer (Pet) (FIG. 1E).

Specific steps: ① 289 mg of L-tyrosine-N-carboxyl cyclic anhydride (L-Tyr-NCAs) and 400 mg of methoxypolyethylene glycol amino (mPEG-NH2) with a molecular weight of 2000 g/moL were weighed and added to a round-bottom flask with magnetic stirring, and 10 mL of anhydrous N,N-dimethylformamide (DMF) was added to fully dissolve; ② the above mixture was placed in a water bath at 30° C and reacted for 72 hours under nitrogen flow to obtain a methoxypolyethylene glycol-poly L-tyrosine (PETyr) block copolymer; ③ The PETyr solution was added dropwise to 200 mL of ice ether for precipitation, suction-filtered under reduced pressure, and dried in a vacuum drying oven to constant weight to obtain PETyr lyophilized powder; ④ The PETyr lyophilized powder was dissolved in water, packaged in a dialysis bag for dialysis for a period of 48 hours, with the molecular weight cutoff of the dialysis bag of 1,000 Da, then freeze-dried at a freezing temperature of -50° C to obtain the copolymer PETyr; ⑤ PETyr (1 g) was reacted with tyrosinase (1 KU) at room temperature for 48 hours to obtain the oxidized methoxypolyethylene glycol-poly L-tyrosine copolymer (Pet).

Then, mechanical characterization was carried out using an AR 2000ex rheometer (TA). The results of rheological experiments showed that in the angular frequency range of 0.1-100 rad/s, the Pet storage energy (G') was always greater than its loss modulus (G") under different time and strain tests, indicating that the prepared Pet had good stability and gelling properties (FIGS. 1D, F, H). The secondary structure of the polypeptides in the hydrogel was analyzed by circular dichroism (J-810, JASCO), which indicated that the secondary structure of Pet had typical characteristic peaks (FIG. 1G). The vial flip test showed that the hydrogel was successfully prepared. The internal morphology of the hydrogel was studied by scanning electron microscopy (SEM; S-4800, Japan), which showed that Pet had a porous network structure (FIG. 1I).

### 3. Preparation of exosome polytyrosine polypeptide hydrogel

The extracted mesenchymal stem cell exosomes were evenly dispersed into the oxidized methoxypolyethylene glycol-poly L-tyrosine amphiphilic copolymer (Pet) solution prepared in the above step 2 at room temperature, with a mass ratio of 1:1, and the mixture was mixed by oscillating on an oscillator to obtain the exosome polytyrosine polypeptide hydrogel.

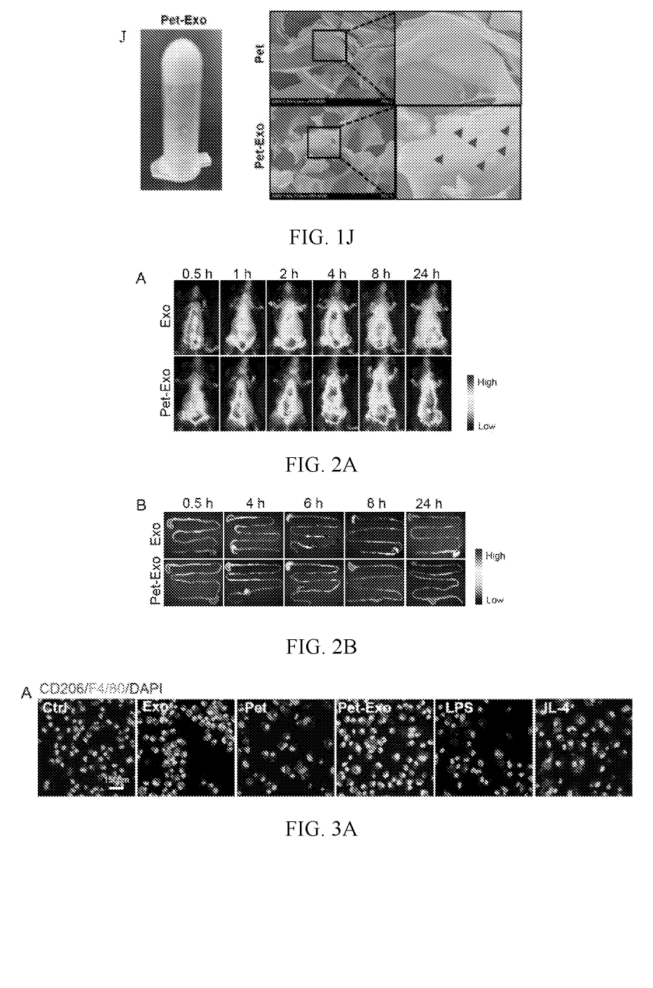

After inversion, the hydrogel did not flow, and the hydrogel state of Pet was not changed. The results of rheological experiments showed that the storage energy (G’) of Pet-Exo was always greater than its loss modulus (G”) at different times, and Exo did not affect the gelling properties of Pet (FIGS. 2D, H). In addition, the scanning electron microscopy (SEM) showed that Exo was evenly distributed in the Pet microporous network structure and had little effect on the structural morphology of the hydrogel (FIG. 2J).

Example 2 In vivo biodistribution of exosome polytyrosine polypeptide hydrogel

In this study, 6-8 week-old C57BL/6J male mice (Beijing HFK Bioscience Co., Ltd.) were used to construct a radiation-induced intestinal injury model. Near-infrared dye (NIR) labeled Exo and Pet-Exo (both at a concentration of 100 mg/mL) were administered once orally at a dose of 200 μL/time. After administration, the biodistribution of NIR fluorescence signals was analyzed at different time points using a small animal in vivo imaging system.

0.5 hour after oral gavage, NIR signals were detected in the exosomes and exosomes encapsulated by hydrogels in vivo. The highest NIR signal was detected in the exosome alone group at 4 h, which then began to weaken (FIG. 2A). In the hydrogel-encapsulated exosome group, the exosome NIR signal intensity measured at 8 h and 24 h was higher than that of the exosome alone group. In order to further evaluate the intestinal distribution of oral Pet-Exo, the mice were dissected and the intestinal fluorescence imaging was performed at 0.5 h, 4 h, 6 h, 8 h, and 24 h after administration. The results showed that the fluorescence signal of the hydrogel-encapsulated exosome group was significantly stronger than that of the exosome alone group over time (FIG. 2B). This indicated that the hydrogel had good gastrointestinal adaptability to exosomes and prolonged their retention time in the intestine, achieving the effect of sustained release of exosomes.

Example 3 Regulatory (reprogramming) effect of exosomes on macrophages

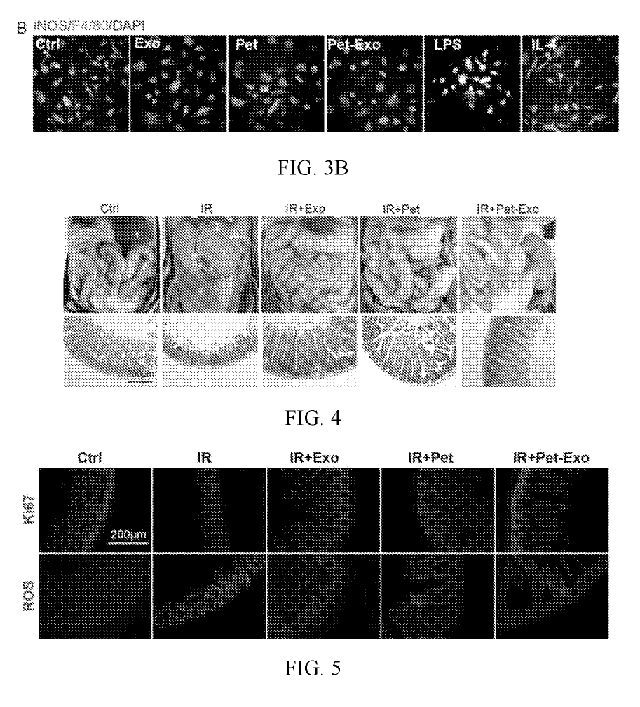

To verify the reprogramming effect of exosomes on macrophages, we first extracted and induced mouse bone marrow-derived macrophages (BMDM), and incubated them with Exo, Pet, Pet-Exo, LPS (M1 macrophage positive control group), and IL-4 (M2 macrophage positive control group) in an incubator at 37° C, 95 % air, and 5 % CO₂ for 48 hours. The immunofluorescence staining was used to detect the expression of CD206 (M2 macrophage marker protein) and iNOS (M1 macrophage marker protein) of macrophages in each group. The results showed that Exo and Pet-Exo promoted the polarization of macrophages toward the anti-inflammatory M2 type (FIG. 3).

Example 4 Pet-Exo effectively reduced the intestinal injury in mice after irradiation

In this study, 6-8 week-old C57BL/6J male mice (purchased from Beijing HFK Bioscience Co., Ltd.) were used to construct a radiation-induced intestinal injury model. The mice were divided into five groups, with 6 mice in each group. The untreated group (Ctrl group) was the negative control group, the cesium source irradiation group (IR group) was the positive control group, and the IR+Exo group, IR+Pet group, and IR+Pet-Exo group were the experimental groups (among which, the dosage and method of administration in the three experimental groups were the same). The specific experimental process was as follows: Pet-Exo (100 mg/mL, 200 μL each time) was administered to mice by oral gavage 48 h and 24 h before abdominal irradiation. Subsequently, the abdomen of the mice was locally irradiated (total dose of 16 Gy) using a cesium source (dose rate of 0.8 Gy/min), and a lead shielding device was used to protect other parts of the mice during the irradiation process to avoid damage to the hematopoietic system. After irradiation, the mice were administered twice every other day at the same time point according to the dosage described above. After the last administration, the mice were killed by cervical dislocation and intestinal tissue samples were collected to complete the establishment of the radiation-induced intestinal injury model and the evaluation of the therapeutic effect of Pet-Exo. The key pathological characteristics of intestinal tissue injury included mucus layer destruction, epithelial cell damage, and inflammatory cell infiltration, and the like. The abdominal cavity of the mice was dissected before sampling, and the intestines of mice in each group were observed. There were ulceration and inflammation on the intestinal surfaces in the IR group, while there was no obvious inflammation in the treatment group. After sampling, the intestines of mice were subjected to histopathological analysis in each group. The results of HE staining showed that the intestinal epithelial structure and intestinal villi were intact and the crypt structure was clear in the Pet-Exo group, which alleviated the phenomena of intestinal villus loss and incomplete epithelial structure caused by IR (FIG. 4). The above results showed that oral administration of Pet-Exo could improve the intestinal injury caused by radiation.

Example 5 Pet-Exo promoted the proliferation of intestinal cells and reduced oxidative damage

The immunofluorescence staining of Ki67 and ROS was performed on intestinal tissue sections of mice in each group. The results showed that the Ki67 expression was up-regulated and the ROS expression was down-regulated in the Pet-Exo group (FIG. 5). This indicated that Pet-Exo promoted the proliferation of intestinal cells after radiation damage and reduced the oxidative damage possibly caused by the increase of radiation-induced ROS.

Example 6 Pet-Exo improved the repair of intestinal injury and the immune regeneration microenvironment

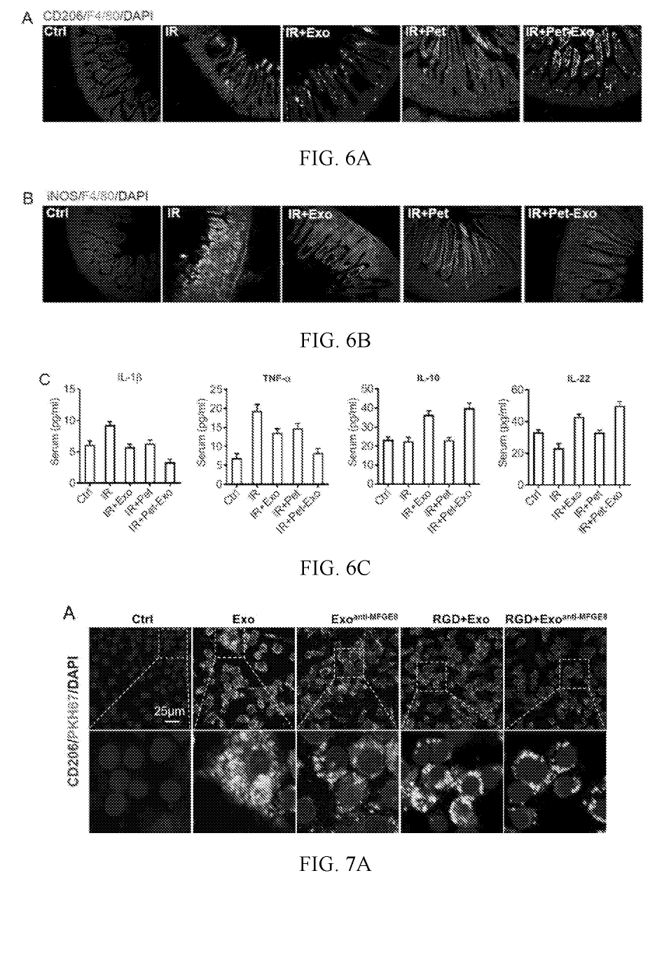

Immunofluorescence results of mouse intestinal tissues showed that Pet-Exo promoted the polarization of M2 macrophages (CD206) and inhibited the expression of iNOS associated with M1 macrophages, thereby improving the immune regeneration microenvironment and promoting the injury repair (FIG. 6). The results of ELISA detection of the expression of inflammation-related cytokines in the serum of mice showed that the pro-inflammatory cytokines (IL-1β and TNF-α) were down-regulated and the anti-inflammatory cytokines (IL-22 and IL-10) were up-regulated in the Pet-Exo group (FIG. 6), indicating that Exo effectively exerted an anti-inflammatory effect. This effect was related to the polarization of macrophages. The above results showed that Pet-Exo inhibited the polarization of macrophages into M1 type and promoted the reprogramming of macrophages into M2 type, thereby exerting an anti-inflammatory effect.

Example 7 Exosomes regulated the macrophage polarization via the MFGE8-ERK/STAT3 axis

The exosome membrane contains MFGE8. Integrin αvβ3 is a transmembrane receptor widely expressed on the surface of macrophages and is involved in cell adhesion, migration and signal transduction. Studies have shown that MFGE8 can specifically bind to αvβ3 through its RGD sequence, triggering the activation of downstream signaling pathways and thus regulating cell function. Therefore, we used MFGE8 neutralizing antibodies to block MFGE8 on the surface of Exo. Meanwhile, the exogenous RGD was used to competitively bind to αvβ3 on the surface of macrophages. The interaction between Exo (PHK67 staining) and M2 macrophages was observed to detect whether Exo could also promote the reprogramming of macrophages into M2 type.

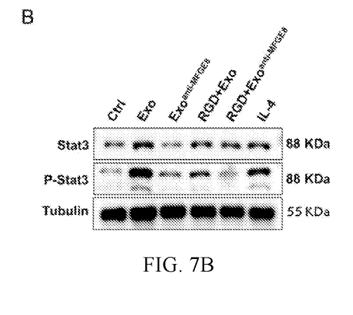

The results of immunofluorescence staining showed that, compared with the Exo-treated group, the MFGE8 neutralizing antibody group that blocked the Exo membrane and the RGD-treated group that competitively bound to αvβ3 on the surface of macrophages showed a significant decrease in M2 macrophages, and the combined treatment group showed a more obvious decrease in M2 macrophages (FIG. 7A).

In order to explore the signaling pathway by which Exo promotes macrophage reprogramming by binding to αvβ3 on the surface of macrophages through MFGE8, combining with the previous transcriptome sequencing analysis of intestinal tissues of mice in each group, we performed Western Blot experiments on the differentially enriched signaling pathways. The results showed that the levels of phosphorylated ERK and STAT3 in the MFGE8 neutralizing antibody group, the RGD blocking macrophage surface αvβ3 receptor group and the combination group were significantly lower than those in the Exo group, which were more obvious in the combination group (FIG. 7B). The above results indicated that the binding of Exo membrane protein MFGE8 to αvβ3 on the surface of macrophages could significantly activate the ERK/STAT3 signaling pathway, which played a crucial role in regulating the M2 polarization of macrophages. However, there are currently no reports on the role of the MGFGE8-ERK axis in the M2 polarization of macrophages. Therefore, in this study, MGFGE8-knockout MSC exosomes and αvβ3-knockout macrophages would be further constructed and the ERK inhibitors would be used to verify the effects on ERK phosphorylation and M2 markers (Arg-1 and CD206).

The foregoing embodiments described are only preferred embodiments and are not intended to limit the scope of the present disclosure. Various modifications and improvements made to the technical solutions of the present disclosure by persons of ordinary skill in the art without departing from the design spirit of the present disclosure shall fall within the scope of protection of the present disclosure defined by appended claims herein.

1. A method for preparing an exosome polypeptide hydrogel, comprising the following steps:
S1, obtaining an exosome solution;
S2, preparation of an oxidized methoxypolyethylene glycol-poly L-tyrosine copolymer Pet: dissolving mPEG-NH₂ in anhydrous DMF under protection of inert gas, then adding L-Tyr-NCAs, reacting at 30-50°C for 48-96 hours, after precipitation, filtration, and drying, adding water to dissolve, then dialyzing in water for 24-72 hours using a dialysis bag, freeze-drying after dialysis to obtain a copolymer PETyr, reacting the copolymer with tyrosinase for 24-96 hours to obtain Pet;
S3, mixing the exosome solution obtained in the step S1 with the Pet in the step S2 in a ratio of 1-2:1-2 to obtain an exosome polypeptide hydrogel.

2. The method according to claim 1, wherein an exosome in the exosome solution in the step S1 is derived from mesenchymal stem cells.

3. The method according to claim 2, wherein the exosome concentration in the exosome solution is 3 mg/mL.

4. The method according to claim 1, wherein the inert gas in the step S2 is argon.

5. The method according to claim 1, wherein the step S2 comprises: dissolving 400 mg of mPEG-NH₂ in 10 mL of anhydrous DMF, then adding 289 mg of L-Tyr-NCAs, reacting at 30°C for 72 hours, after precipitation, filtration, and drying, adding water to dissolve, then dialyzing for 24-72 hours using a dialysis bag with a molecular weight cutoff of 1,000-2,000 Da, freeze-drying to obtain a copolymer PETyr, reacting the copolymer with tyrosinase for 48 hours to obtain Pet.

6. The method according to claim 5, wherein a molecular weight of the mPEG-NH2 is 2000 g/moL.

7. The method according to claim 6, wherein a molecular weight cutoff of the dialysis bag is 1,000 Da and a dialysis time is 48 hours.

8. The method according to claim 1, wherein a dosage ratio of the exosome solution to the Pet in the step S3 is 1:1.

9. An exosome polypeptide hydrogel prepared by the method for preparing an exosome polypeptide hydrogel according to claim 1.

10. An exosome polypeptide hydrogel prepared by the method for preparing an exosome polypeptide hydrogel according to claim 2.

11. An exosome polypeptide hydrogel prepared by the method for preparing an exosome polypeptide hydrogel according to claim 3.

12. An exosome polypeptide hydrogel prepared by the method for preparing an exosome polypeptide hydrogel according to claim 4.

13. An exosome polypeptide hydrogel prepared by the method for preparing an exosome polypeptide hydrogel according to claim 5.

14. An exosome polypeptide hydrogel prepared by the method for preparing an exosome polypeptide hydrogel according to claim 6.

15. An exosome polypeptide hydrogel prepared by the method for preparing an exosome polypeptide hydrogel according to claim 7.

16. An exosome polypeptide hydrogel prepared by the method for preparing an exosome polypeptide hydrogel according to claim 8.

17. Use of the exosome polypeptide hydrogel of claim 9 in the preparation of a product for treating radiation-induced intestinal injury.

18. Use of the exosome polypeptide hydrogel of claim 10 in the preparation of a product for treating radiation-induced intestinal injury.

19. Use of the exosome polypeptide hydrogel of claim 11 in the preparation of a product for treating radiation-induced intestinal injury.

20. Use of the exosome polypeptide hydrogel of claim 12 in the preparation of a product for treating radiation-induced intestinal injury.
